(19) United States
(12) Patent Application Publication
(10) Pub. No.: US 2017/0319084 A1
(43) Pub. Date: Nov. 9, 2017

(54) MEASURING APPARATUS AND MEASURING METHOD

(71) Applicant: KYOCERA Corporation, Kyoto (JP)

(72) Inventor: Takuya FUJIWARA, Yokohama-shi, Kanagawa (JP)

(73) Assignee: KYOCERA Corporation, Kyoto (JP)

(21) Appl. No.: 15/526,893

(22) PCT Filed: Nov. 11, 2015
(86) PCT No.: PCT/JP2015/005623

§ 371 Date: May 15, 2017

(30) Foreign Application Priority Data
Nov. 18, 2014 (JP) 2014-233744

(51) Int. Cl. A61B 5/026; G01S 7/52; A61B 8/08; A61B 5/02; A61B 8/13; G01S 15/89; G01F 1/66; A61B 8/00

(52) U.S. Cl. CPC A61B 5/0261; G01S 15/8979; G01S 7/52073; G01F 1/663; A61B 8/0858; A61B 5/02007; A61B 8/13; A61B 8/461; G01N 2291/02836

(57) ABSTRACT

A measuring apparatus configured to measure the biological information includes a light emitter configured to emit measuring light, a light receiver including a plurality of light receiving areas that receive scattering light of the measuring light from a measured part, and a controller configured to generate the biological information based on output from the plurality of light receiving areas.

### CROSS-REFERENCE TO RELATED APPLICATION

This application claims priority to and the benefit of Japanese Patent Application No. 2014-233744 filed on Nov. 18, 2014, the entire contents of which are incorporated herein by reference.

### TECHNICAL FIELD

The present disclosure relates to a measuring apparatus and a measuring method.

### BACKGROUND

A measuring apparatus is known which obtains the biological output information from a measured part such as a fingertip of a subject (user).

### SUMMARY

A measuring apparatus configured to measure the biological information according to the present disclosure includes:
a light emitter configured to emit measuring light;
a light receiver including a plurality of light receiving areas that receive scattering light of the measuring light from a measured part; and
a controller configure to generate the biological information based on output from the plurality of light receiving areas.

Further, it is to be understood that the present disclosure can be achieved as a method corresponding substantially to the above described measuring apparatus, and the method is included in the scope of the present disclosure.

For example, a measuring method according to the present disclosure includes the steps of:
emitting measuring light by a light emitter;
receiving scattering light of the measuring light from a measured part by a light receiver including a plurality of light receiving areas; and
generating the biological information by a controller based on the output from the plurality of light receiving areas.

### BRIEF DESCRIPTION OF THE DRAWINGS

In the accompanying drawings:

FIG. 1 is a functional block diagram illustrating a schematic configuration of a measuring apparatus according to Embodiment 1 of the present disclosure;

FIG. 2 is a diagram illustrating an example of the measuring apparatus in a use state;

FIG. 3 is a schematic diagram illustrating an example of arrangement of light receiving areas in a light receiver illustrated in FIG. 1;

FIG. 4 is a conceptual diagram of a main part illustrating an example of a method of measuring the biological information by the measuring apparatus illustrated in FIG. 1;

FIG. 5 is a flowchart illustrating an example of a process by the measuring apparatus according to Embodiment 1;

FIG. 6 is a functional block diagram illustrating a schematic configuration of a measuring apparatus according to Embodiment 2 of the present disclosure;

FIG. 7 is a diagram illustrating an example of blood flow distribution generated by a controller of the measuring apparatus according to Embodiment 2;

FIG. 8 is a flowchart illustrating an example of a process by the measuring apparatus according to Embodiment 2;

FIGS. 9A and 9B are diagrams illustrating arrangement variations of the light receiving areas; and

FIGS. 10A and 10B are diagrams illustrating an example of a cellular phone with the measuring apparatus illustrated in FIG. 1 or FIG. 6.

### DETAILED DESCRIPTION

The biological information measured by the measuring apparatus is variable depending, for example, on the pressing conditions such as the pressing force from the measured part to the measuring apparatus and the pressed position on the measuring apparatus. As a result, in the conventional measuring apparatus, the measurement accuracy of the biological information is likely to be decreased. Further, in the case of authentication using the biological information, if the measurement accuracy of the biological information is low, the authentication accuracy decreases.

It would therefore be helpful to provide a measuring apparatus and a measuring method capable of improving the measurement accuracy of the biological information.

The disclosed embodiments will be described in detail below with reference to the drawings.

### Embodiment 1

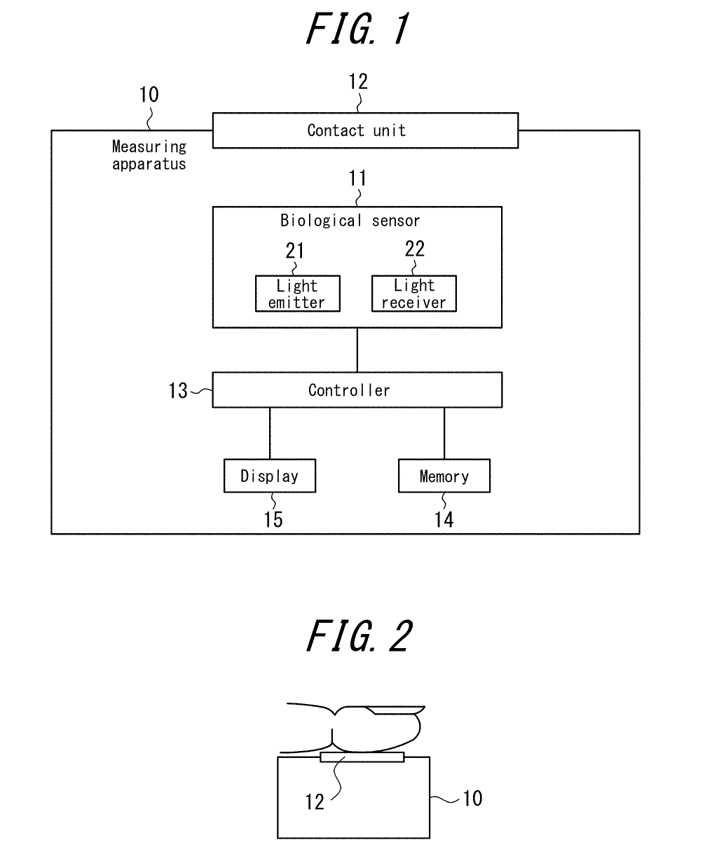

FIG. 1 is a functional block diagram illustrating a schematic configuration of a measuring apparatus according to Embodiment 1 of the present disclosure. The measuring apparatus 10 includes a biological sensor 11, a contact unit 12, a controller 13, a memory 14 and a display 15.

The measuring apparatus 10 measures the biological information in the measured part being in contact with the contact unit 12. FIG. 2 is a diagram illustrating an example of the measuring apparatus 10 in a use state, and illustrates a state where the user pushes the measuring apparatus 10 with his/her finger, which is a measured part. The measuring apparatus 10 measures the biological information with a finger pressed to the contact unit 12 as illustrated in FIG. 2. The biological information may be any biological information that can be measured by using the biological sensor 11. In this embodiment, the measuring apparatus 10 is described below as an example assuming that it measures blood flow rate, which is the information regarding the blood flow, of the subject.

In FIG. 1, the biological sensor 11 obtains the biological information from the measured part. As this embodiment, when the measuring apparatus 10 measures the blood flow rate, the biological sensor 11 includes a light emitter 21 and a light receiver 22.

The light emitter 21 emits laser light based on control by the controller 13. The light emitter 21 radiates, as measuring light, laser light with a wavelength at which a predetermined component contained in the blood can be detected, for example, to the measured part, and is configured with a laser diode (LD), for example.

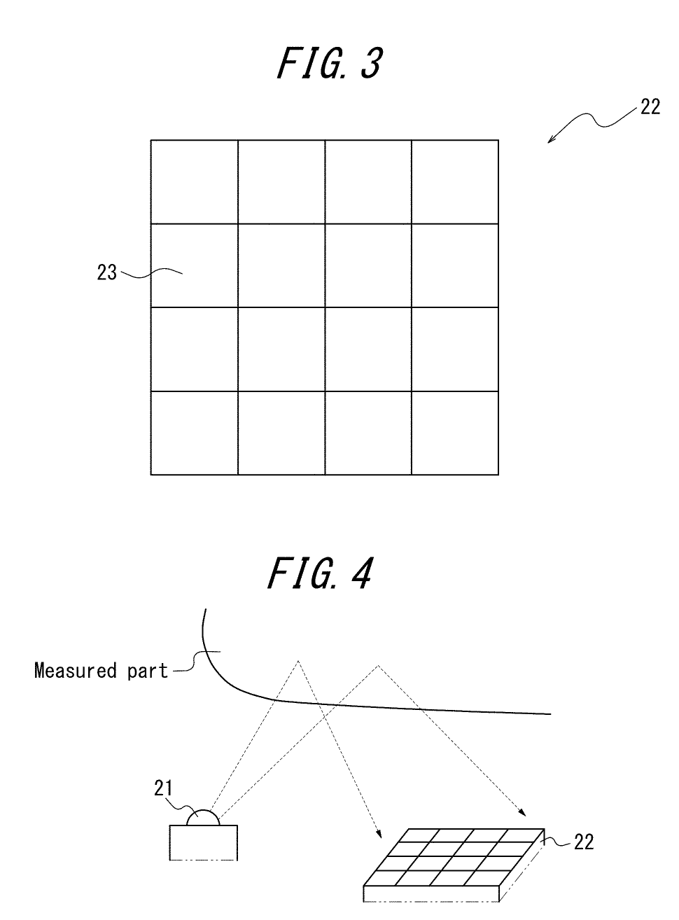

The light receiver 22 receives, as the biological information, scattering light of the measuring light from the measured part. The light receiver 22 has a plurality of light receiving areas and receives scattering light in each light receiving area. FIG. 3 is a schematic diagram illustrating an example of arrangement of the light receiving areas in the light receiver 22 illustrated in FIG. 1. In this embodiment, the light receiver 22 has light receiving areas 23 arranged in a grid formed by 16 squares in total with 4 rows and 4 columns. For example, photodiode (PD) is disposed in each light receiving area 23. The biological sensor 11 sends photoelectric conversion signal (biometric output) of the scattering light received in each light receiving area 23 of the light receiver 22 to the controller 13. As for the light receiver 22, an imaging lens may be disposed on the side of its light receiving surface where the scattering light is received so that an image regarding the biological information in the measured part can be formed.

FIG. 4 is a conceptual diagram of the main portion illustrating an example of a method of measuring the biological information by the measuring apparatus 10 illustrated in FIG. 1. The measuring light emitted by the light emitter 21 is scattered by the component flowing through the blood vessel of the measured part, and the scattering light from the measured part is received in each light receiving area 23 of the light receiver 22. Thus, when a plurality of light receiving areas 23 are arranged on the light receiver 22, each light receiving area 23 receives scattering light from different positions of the measured part.

In FIG. 1, the contact unit 12 is a portion touched by a subject with his/her measured part such as a finger in order to measure the biological information. The contact unit 12 is configured with a member in the form of plate, for example. The contact unit 12 is configured with a member transparent at least to measuring light from the light emitter 21 and scattering light from the measured part.

The controller 13 is a processor for controlling the entire measuring apparatus 10 including each functional block of the measuring apparatus 10. The controller 13 is configured with of a processor such as a central processing unit (CPU) that executes a control procedure program, and such program is stored in the memory 14, an external storage medium, or the like.

The controller 13 controls emission of laser light from the light emitter 21. Upon being ready to measure the biological information by subject's operation, for example, the controller 13 causes the light emitter 21 to emit laser light. The measuring apparatus 10 includes a detector configured to detect contact of the measured part with the contact unit 12. When determining that the measured part touches the contact unit 12 based on the output from the detector, the controller 13 may cause the light emitter 21 to emit laser light. Upon emission of the laser light, the biological sensor 11 starts obtaining the biological information.

After the biological sensor 11 starts obtaining the biological information by emission of the laser light, the controller 13 determines whether obtaining the biological information by the biological sensor 11 is finished or not. Upon determining that the obtaining the biological information is finished, the controller 13 causes the light emitter 21 to stop outputting the laser light. The controller 13 may determine that the obtaining the biological information is finished in a predetermined period of time after the biological sensor 11 starts obtaining the biological information, for example. Further, the controller 13 may determine that the obtaining the biological information is finished when the biological sensor 11 obtains the biological information sufficient to measure the biological information. As described above, the controller 13 controls obtaining the biological information by the biological sensor 11.

When the obtaining of biometric output by the biological sensor 11 is finished, the controller 13 generates the biological information based on the biometric output from the biological sensor 11. It is noted that, as the light receiver 22 has a plurality of light receiving areas 23, the biometric output from the biological sensor 11 includes output regarding to the intensity of the scattering light received by each of the plurality of light receiving areas 23.

In this embodiment, the controller 13 calculates the biological information (biological information candidate) measured in each light receiving area 23 based on the output regarding the intensity of the scattering light received in each of the plurality of light receiving areas 23 (16 areas in this embodiment). The biological information candidate is calculated as blood flow rate measured in each light receiving area 23 of the light receiver 22. That is, in this embodiment, 16 blood flow rates are calculated as the biological information candidate.

The blood flow rate measurement technique using Doppler shift employed by the controller 13 will be described below. When measuring the blood flow rate, the controller 13 causes the light emitter 21 to radiate the laser light into body tissue (examined part) and the scattering light scattered from the body tissue is received by the light receiver 22. Then the controller 13 calculates the blood flow rate based on the output regarding the received scattering light.

In the body tissue, the scattering light scattered from the moving blood cell is subject to the frequency shift (Doppler shift) due to Doppler effect that is proportional to the transfer rate of blood cells in the blood. The controller 13 detects beat signals resulting from interference of the scattering light from the static tissue and that from the moving blood cells. The beat signal represents the intensity as a function of time. Then the controller 13 converts the beat signal into power spectrum representing power as a function of frequency. In this power spectrum of the beat signal, Doppler shift frequency is proportional to the blood cell velocity, and the power corresponds to the blood cell volume. Then the controller 13 multiplies the power spectrum of the beat signal with the frequency and integrates to find the blood flow rate.

The controller 13 generates a piece of biological information based on a comparison of calculated 16 biological information candidates. For example, the controller 13 selects the most suitable biological information candidate from the blood flow rate, which is the calculated 16 biological information candidates, and determines the selected biological information candidate as the blood flow rate, which is the biological information of the subject, thereby a piece of biological information is generated. The controller 13 can select the most suitable biological information candidate by any appropriate method. For example, the controller 13 selects, among the calculated 16 biological information candidates, the biological information candidate calculated based on output from the light receiving area 23 corresponding to the area of the contact unit 12 pressed by the measured part with a pressure most suitable to measure the biological information as the most suitable biological information candidate. The light receiving area 23 corresponding to the area of the contact unit 12 pressed by the measured part with a pressure most suitable to measure the biological information is an area where change per one pulse of the subject relative to the intensity of the scattering light received in each light receiving area 23 is the largest, and is determined by the controller 13. That is, the controller 13 generates, as the biological information, the biological information candidate calculated based on the biometric output in the light receiving area 23 where change in the intensity of the received scattering light is the largest. The measured part such as a finger is not a flat surface, thus the pressure applied from the measured part to the contact unit 12 changes depending on the area of the contact unit 12. In the measuring apparatus 10 according to this embodiment, as described above, the controller 13 generates, as the biological information of the subject, the biological information obtained in the area of the contact unit 12 pressed with a pressure most suitable to measure the biological information. Thus, the measuring apparatus 10 according to this embodiment has an improved measurement accuracy of the biological information and a higher reliability of test results compared to the measuring apparatus that obtains only a piece of biological information by the biological sensor 11.

The controller 13 displays the generated biological information on the display 15. The subject may know the blood flow rate by confirming the displayed measurement results.

The memory 14 may be configured with a semiconductor memory or a magnetic memory or the like, store a variety of information and a program for operating the measuring apparatus 10 or the like, and serve also as a work memory. The memory 14 stores the information regarding the arrangement of each light receiving area 23, for example.

The display 15 is a display device such as a liquid crystal display, an organic EL display or an inorganic EL display. For example, the display 15 displays the measurement results of the biological information by the measuring apparatus 10.

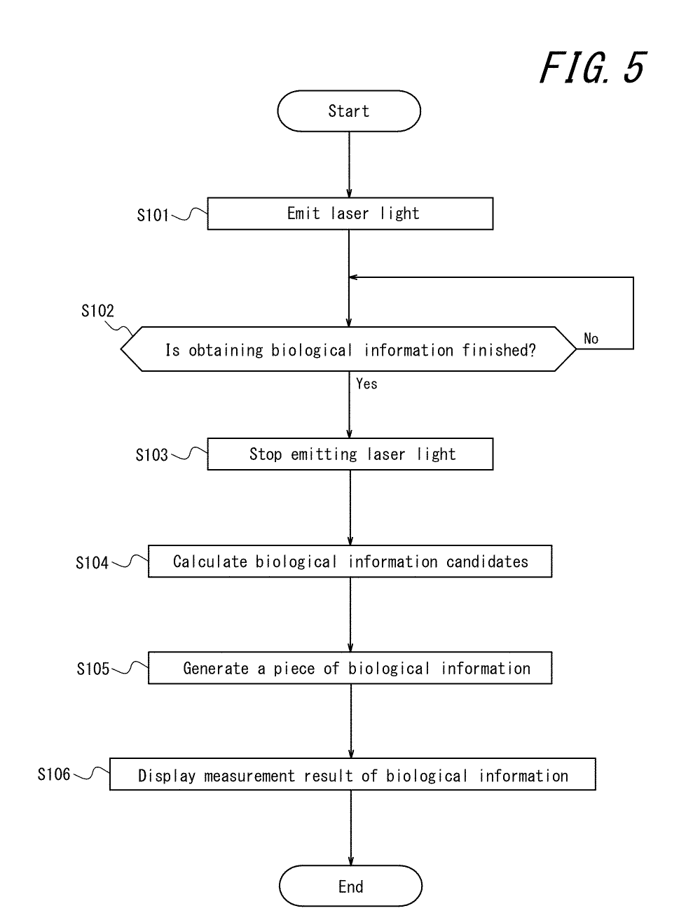

Next, an example of the measurement process of blood flow rate performed by the measuring apparatus 10 according to Embodiment 1 will be described with reference to the flowchart illustrated in FIG. 5. Upon being ready to measure the biological information by subject's operation, for example, the measuring apparatus 10 starts the flow illustrated in FIG. 5.

First, the controller 13 causes the light emitter 21 to emit laser light (step S101). When laser light is emitted, the controller 13 causes the biological sensor 11 to start obtaining the biological information. When the biological sensor 11 obtains the biological information, the controller 13 obtains, from the biological sensor 11, the biometric output that includes output regarding the intensity of the scattering light received by each of the plurality of light receiving areas 23 and stores the obtained biometric output in the memory 14.

The controller 13 determines whether obtaining the biological information by the biological sensor 11 is finished or not (step S102).

Upon determining that obtaining the biological information by the biological sensor 11 is not finished (No in step S102), the controller 13 repeats step S102 until it determines that obtaining the biological information is finished.

Upon determining that obtaining the biological information by the biological sensor 11 is finished (Yes in step S102), the controller 13 causes the light emitter 21 to stop emitting the laser light (step S103).

Then the controller 13 calculates 16 biological information candidates corresponding to each light receiving area 23 based on the biometric output obtained and stored in the memory 14 (step S104).

The controller 13 generates a piece of biological information based on a comparison of the calculated 16 biological information candidates (step S105).

The controller 13 displays measurement results of the biological information on the display 15 (step S106). The subject may know the blood flow rate by confirming the displayed measurement results.

In this way, in the measuring apparatus 10 according to Embodiment 1, the controller 13 calculates the biological information candidate with respect to each piece of biological information obtained in each light receiving area 23 of the light receiver 22, and generates a piece of biological information based on a comparison of calculated biological information candidates. Thus, in the measuring apparatus 10, the measurement accuracy of the biological information can be improved compared to the measuring apparatus in which only a piece of biological information is obtained by the biological sensor 11.

### Embodiment 2

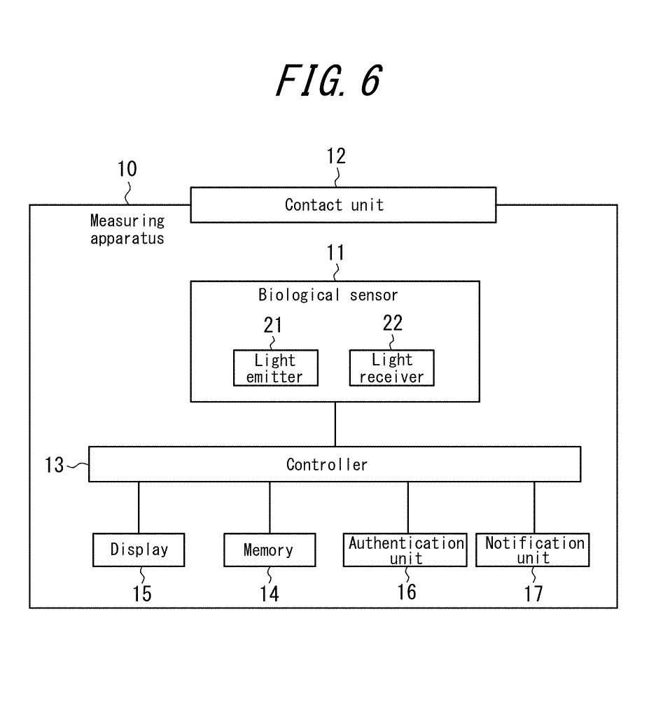

FIG. 6 is a functional block diagram illustrating a schematic configuration of the measuring apparatus 10 according to Embodiment 2 of the present disclosure. In Embodiment 2, the measuring apparatus 10 further includes an authentication unit 16 and a notification unit 17. The measuring apparatus 10 according to Embodiment 2 generates the biological information and after that, authenticates the subject by using the generated biological information. The measuring apparatus 10 according to this embodiment can improve the authentication accuracy through improvement of the measurement accuracy of the biological information. Description of the points that are the same as those of Embodiment 1 will be omitted, and different points will be described below.

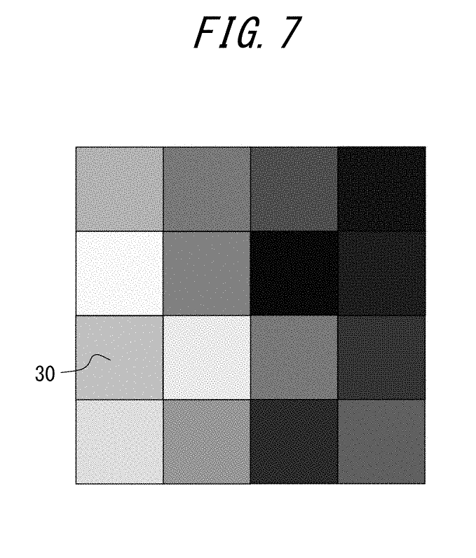

In Embodiment 2, the controller 13 generates a plurality of distributions regarding the biological information (biological information distribution) as the biological information. In this embodiment, the biological sensor 11 measures the information regarding the blood flow. The biological information distribution according to this embodiment refers in particular to the blood flow distribution. The blood flow distribution is a distribution of the intensity of scattering light based on the blood flow received by each light receiving area 23 at a specific point of time, and is represented by gray scale images, for example, as schematically illustrated in FIG. 7. The controller 13 generates the blood flow distribution at regular time intervals based on the time stamp function, for example.

The 16 areas (blood flow distribution areas) 30 illustrated in the blood flow distribution in FIG. 7 correspond respectively to each light receiving area 23 of the light receiver 22. As the intensity of the scattering light received by the light receiving area 23 corresponding to the blood flow distribution area 30 increases, the brightness of the blood flow distribution area 30 displayed on the blood flow distribution increases. Meanwhile, as the intensity of the scattering light received by the light receiving area 23 decreases, the brightness of the blood flow distribution area 30 displayed on the blood flow distribution decreases. As the measuring light is scattered by blood cells in the body tissues, the intensity of the scattering light changes depending on the amount of blood cells when the light is received. As each light receiving area 23 receives the scattering light from different positions on the measured part, each blood flow distribution area 30 of blood flow distribution indicates the information regarding the blood flow on different positions of the measured part. As described above, in this embodiment, the controller 13 generates the blood flow distribution in the measured part as the biological information.

Further, the controller 13 analyzes the tendency of change in the generated plurality of blood flow distributions. As the blood flow distribution is an intensity distribution of the scattering light based on the blood flow at a specific point of time, blood cells in the tissue move as the blood flow flows over time, and as a result, the blood flow distribution changes. The controller 13 analyzes such tendency of change in the blood flow distribution. The controller 13 specifies an area whose brightness is higher than a predetermined brightness in each blood flow distribution area 30 of blood flow distribution, for example. The area whose brightness is higher than a predetermined brightness indicates that, in the area of the measured part measured by the light receiving area 23 corresponding to the above mentioned area, the blood flow rate is larger than the predetermined flow. The controller 13 analyzes change in the quantity, position, or the like, of the area whose brightness is higher than the predetermined brightness in a plurality of blood flow distributions to determine the tendency of change in the blood flow distribution. Such tendency of change reflects the blood flow rate flowing through the blood vessels, and thus the controller 13 can estimate a local blood flow direction based on the tendency of change.

The subject stores in advance the tendency of change in the memory 14 before performing authentication by using the measuring apparatus 10. Specifically, when the subject performs a predetermined operation for storing the tendency of change to the measuring apparatus 10, the measuring apparatus 10 obtains the biological information from the measured part of the subject in contact with the contact unit 12 and generates the biological information distribution. The measuring apparatus 10 analyzes the tendency of change in the generated biological information distribution and causes the memory 14 to store the tendency of change in the biological information distribution of the subject. The measuring apparatus 10 may generate the biological information distribution each time the subject measures the biological information by using the measuring apparatus 10 to analyze the tendency of change thereof, and based on the analyzed tendency of change, update the tendency of change in the biological information distribution stored in the memory 14.

The authentication unit 16 authenticates based on a comparison between the biological information distribution generated by the controller 13 and the biological information distribution of the subject stored in the memory 14. Specifically, in this embodiment, the authentication unit 16 compares the tendency of change analyzed by the controller 13 when authenticating the subject and the tendency of change stored in the memory 14. The authentication unit 16 determines that the authentication is successful when the tendencies of change are the same, and determines that the authentication is unsuccessful when they are different. As is well known in the vein authentication technology or the like, the blood vessel patterns of the measured part differ from subject to subject. Further, the direction of blood flow in the blood vessel is constant in the same subject. As described above, the blood flow distribution and its tendency of change depend on the subject, and are constant in the same subject. Thus, the authentication unit 16 can authenticate the subject based on the blood flow distribution and its tendency of change. It is noted that, in this embodiment, although the authentication unit 16 was described as a function unit independent from the controller 13, the function possessed by the authentication unit 16 may be included in the controller 13.

When the authentication unit 16 determines that the authentication is successful, the controller 13 notifies the information indicating that the authentication is completed (authentication complete information) from the notification unit 17. The notification unit 17 can notify the information in a visual manner such as, for example, images, characters or light emission, in an auditory manner such as sound, or a combination thereof. When the notification unit 17 notifies in a visual manner, it notifies by displaying images or letters on a display device such as the display 15, or the like. The notification unit 17 may notify by causing a light emitting element such as LED to emit light. When the notification unit 17 notifies in an auditory manner, it notifies, as a sound generation device such as, for example, a speaker, the information through output of alarm sound, voice guidance, or the like. Notification performed by the notification unit 17 is not limited to visual or auditory methods, and may be any method recognizable by the subject.

Also, even if the authentication unit 16 determines that the authentication is unsuccessful, the controller 13 notifies the information indicating that the authentication is unsuccessful (error information) from the notification unit 17. Although the error information may be notified in any manner including the above described example, it may preferably be notified in a manner different from that for notifying the authentication complete information so that the subject who recognizes notification from the notification unit 17 can distinguish between the authentication complete information and the error information. For example, the subject can distinguish between the authentication complete information and the error information when the authentication complete information is notified in a visual manner and the error information is notified in an auditory manner. Further, even if both the authentication complete information and the error information are notified in an auditory manner, the subject can distinguish between the authentication complete information and the error information when they are notified with alarming sounds different from each other.

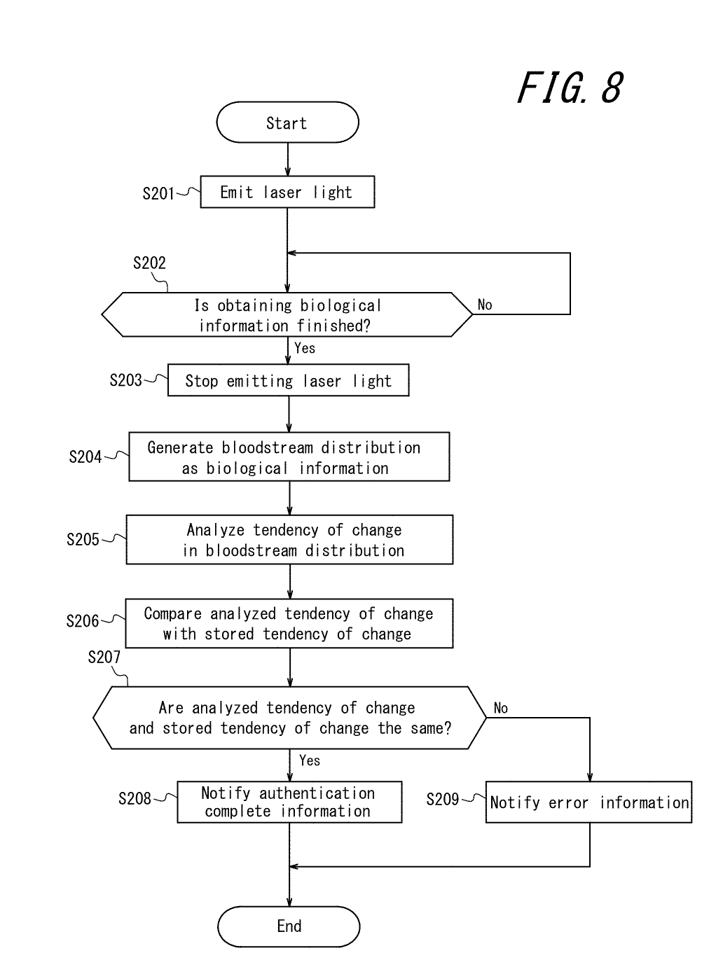

Next, an example of the measurement process of blood flow rate performed by the measuring apparatus 10 according to Embodiment 2 will be described with reference to the flowchart illustrated in FIG. 8. Upon being ready to execute the authentication process by subject's operation, for example, the measuring apparatus 10 starts the flow illustrated in FIG. 8.

From steps S201 to S203, the controller 13 performs the same process as that of the measuring apparatus 10 according to Embodiment 1. That is, the process from steps S201 to S203 corresponds to the process from steps S101 to S103 illustrated in FIG. 5. Thus, description of steps from S201 to S203 will be omitted here.

The controller 13 generates the blood flow distribution as the biological information based on the biometric output obtained from the biological sensor 11 and stored in the memory 14 (step S204). The controller 13 generates the blood flow distribution at regular time intervals based on the time stamp function.

The controller 13 analyzes the tendency of change in the generated plurality of blood flow distributions (step S205).

Next, the authentication unit 16 compares the tendency of change in the blood flow distribution analyzed by the controller 13 and the tendency of change in the blood flow distribution of the subject stored in the memory 14 (step S206).

Then, the authentication unit 16 determines whether the tendency of change in the blood flow distribution analyzed by the controller 13 and the tendency of change in the blood flow distribution stored in the memory 14 are the same or not (step S207).

If the tendency of change in the blood flow distribution analyzed by the controller 13 and the tendency of change in the blood flow distribution stored in the memory 14 are the same (Yes in step S207), the authentication unit 16 determines that the authentication is successful, and the controller 13 notifies the authentication complete information from the notification unit 17 (step S208).

Meanwhile, if the tendency of change in the blood flow distribution analyzed by the controller 13 and the tendency of change in the blood flow distribution stored in the memory 14 are not the same (No in step S207), the authentication unit 16 determines that the authentication is unsuccessful, and the controller 13 notifies the error information from the notification unit 17 (step S209).

In this way, in the measuring apparatus 10 according to Embodiment 2, the controller 13 generates blood flow distribution as the biological information based on the information regarding the blood flow received by the plurality of light receiving areas 23. Then the authentication unit 16 authenticates based on the tendency of change in the blood flow distribution. As the blood flow distribution and its tendency of change depend on the subject and are constant in the same subject, the measuring apparatus 10 can authenticate by using the biological information. Here, the tendency of change is the tendency of change in the blood flow distribution, which is the distribution of intensity of the scattering light based on the blood flow received by the plurality of light receiving areas 23, and as a result, the measuring apparatus 10 can authenticate with an accuracy higher than that of the authentication based on one tendency of change in scattering light. Further, even in the case where the blood flow rate of the subject is temporarily high after exercising, or the like, the blood flow distribution and its tendency of change are constant in the same subject, which allows for accurate authentication. As described above, the measuring apparatus 10 has the light receiver 22 including a plurality of light receiving areas 23, and thus can achieve the functions that cannot be achieved by the measuring apparatus that measures the biological information with one light receiving area.

The present disclosure is not limited only to the above described embodiments, and various changes and modifications are possible. For example, the functions or the like included in each component, each step, or the like, may be reordered in any logically consistent manner. Furthermore, components, steps, or the like, may be combined into one or divided.

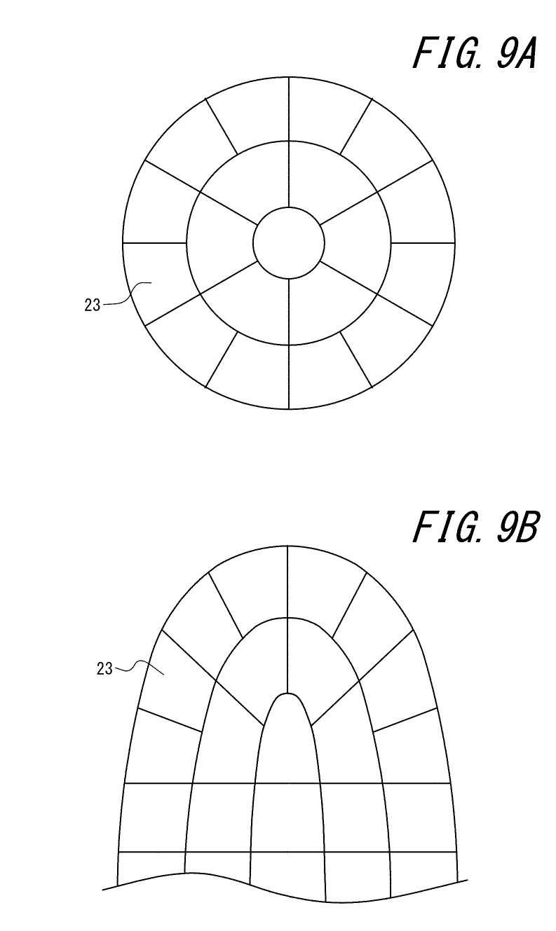

For example, the above described Embodiments 1 and 2 were described assuming that the light receiver 22 has the light receiving area 23 formed by 16 squares arranged in a grid pattern. However, the light receiving area 23 included in the light receiver 22 are not limited to that example. The light receiver 22 may include the light receiving area 23 formed by more than or less than 16 squares, for example. Further, arrangement of the light receiving area 23 is not limited to a grid pattern. For example, as illustrated in FIG. 9(a), the light receiving area 23 may be arranged by appropriately dividing the unit into concentric circles or, as illustrated in FIG. 9(b), it may be arranged by appropriately dividing the unit into areas corresponding to the measured part such as a finger.

Further, based on the result of authentication in Embodiment 2, the measuring apparatus 10 may store the biological information measured by the method described in Embodiment 1 in the memory 14. That is, the measuring apparatus 10 measures the blood flow rate by the method described in Embodiment 1 (the flow illustrated in FIG. 5). Furthermore, the measuring apparatus 10 analyzes the tendency of change in the blood flow distribution by the method described in Embodiment 2 (steps S204 to S206 of the flow illustrated in FIG. 8). Then, the measuring apparatus 10 determines that the blood flow rate of the subject is in a predetermined (healthy or normal) range when it determines that the tendency of change analyzed by the controller 13 and the tendency of change stored in the memory 14 are the same, and stores the measured blood flow rate in the memory 14. Meanwhile, the measuring apparatus 10 determines that the blood flow rate of the subject is not in a predetermined range when it determines that the tendency of change analyzed by the controller 13 and the tendency of change stored in the memory 14 are not the same, and stores the blood flow rate with an error flag, or the like, being associated thereto in the memory 14. As a result of this, in the case where the subject has physical abnormalities or the like, the information regarding the blood flow rate associated with an error flag or the like can be useful for diagnosis of the subject. In this example, when determining that the tendency of change analyzed by the controller 13 and the tendency of change stored in the memory 14 are not the same, the measuring apparatus 10 may notify, from the notification unit 17, that the information indicating that the blood flow rate is associated with an error flag, or the like, which allows the subject to know that the blood flow rate is not in a predetermined range.

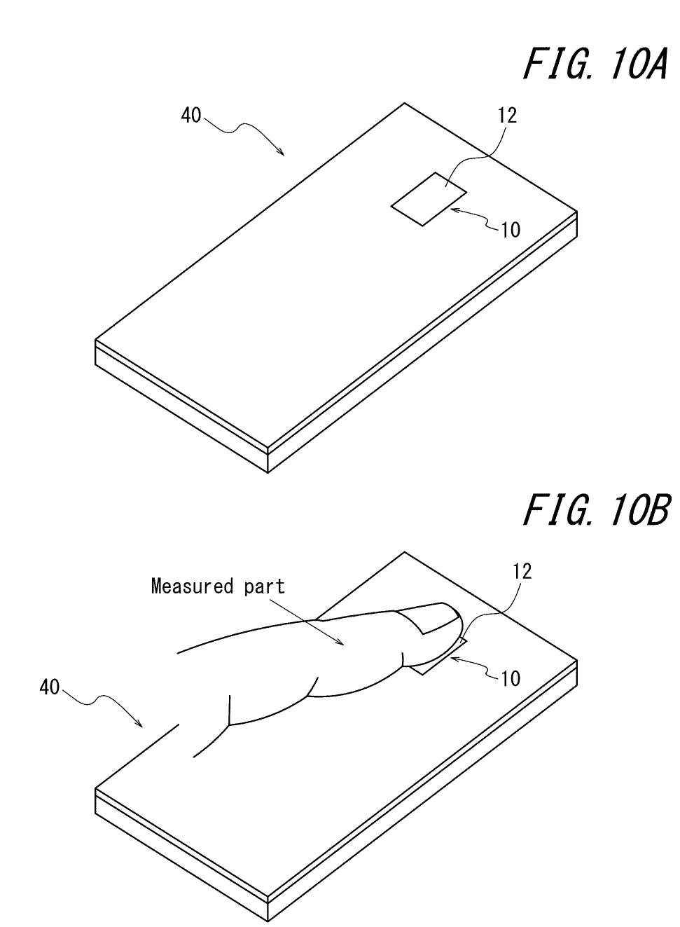

Further, the measuring apparatus 10 according to Embodiments 1 and 2 may be mounted on various electronic devices, for example. FIGS. 10A and 10B are diagrams illustrating an example of a cellular phone with the measuring apparatus 10 illustrated in FIG. 1 or FIG. 6. As illustrated in FIG. 10(a), the cellular phone 40 has the measuring apparatus 10 on its back side. FIG. 10(b) illustrates an example where the user uses the cellular phone 40 with the measuring apparatus 10 to measure the biological information. The user touches the contact unit 12 with his/her finger to cause the measuring apparatus 10 to measure the biological information. When the measuring apparatus 10 according to Embodiment 2 is mounted on the cellular phone 40, the authentication by the measuring apparatus 10 may be functioned as a security lock of the cellular phone 40.

The arrangement of the measuring apparatus 10 in the cellular phone 40 is not limited to that illustrated in FIGS. 10A and 10B. For example, the measuring apparatus 10 may be arranged on other parts on the back of the cellular phone 40, or may be arranged on the surface or the side thereof.

Further, an electronic device mounted with the measuring apparatus 10 is not limited to the cellular phone 40. The measuring apparatus 10 may be mounted on any of a variety of electronic devices such as, for example, a portable music player, a notebook computer, a watch, a tablet terminal, or a game console.

Further, in the above described embodiments, the measuring apparatus 10 was described assuming that it includes the contact unit 12. However, depending on the biological information to be measured, the measuring apparatus 10 may not include the contact unit 12, and may radiate measuring light to the measured part in a non-contact state to measure the biological information.

Further, in the above described Embodiment 1, the controller 13 mounted on the measuring apparatus 10 was described assuming that it generates the biological information based on the output from the light receiver 22. However, the biological information is generated not only by the controller 13 mounted on the measuring apparatus 10. For example, a server apparatus connected to the measuring apparatus 10 over a wired or wireless network, or over a combination of wired and wireless networks may have a function unit corresponding to the controller 13, and the biological information may be generated by the server apparatus that includes the function unit. In this case, the measuring apparatus 10 obtains the biological information from the biological sensor 11, and sends the biometric output based on the obtained biological information from a communication unit provided separately to the server apparatus. Then the server apparatus calculates the biological information candidate with respect to each piece of information obtained in each light receiving area 23 of the light receiver 22, and generates a piece of biological information based on a comparison of the calculated biological information candidates. Then the server apparatus sends the generated biological information to the measuring apparatus 10. When the biological information received by the measuring apparatus 10 is displayed on the display 15, the subject can confirm the measurement results. As described above, when the server apparatus generates the biological information, miniaturization of the measuring apparatus 10 can be achieved compared to the case where all functions illustrated in FIG. 1 are achieved on one measuring apparatus 10.

Further, when the above described server apparatus has a function unit corresponding to the authentication unit 16 according to Embodiment 2, authentication can be performed also by the server apparatus.

1. A measuring apparatus configured to measure biological information, the measuring apparatus comprising:
a light emitter configured to emit measuring light;
a light receiver including a plurality of light receiving areas that receive scattering light of the measuring light from a measured part; and
a controller configured to generate biological information based on output from the plurality of light receiving areas.

2. The measuring apparatus according to claim 1, wherein the controller calculates a plurality of biological information candidates based on the output from the plurality of light receiving areas, and generates a piece of biological information based on a comparison of the plurality of biological information candidates.

3. The measuring apparatus according to claim 1, wherein the controller generates distribution regarding the biological information as the biological information based on the output from the plurality of light receiving areas.

4. The measuring apparatus according to claim 3, further comprising:
a memory configured to store the distribution regarding the biological information of a subject; and
an authentication unit, wherein
the authentication unit authenticates the subject based on a comparison between the distribution regarding the biological information generated by the controller and the distribution regarding the biological information of the subject stored in the memory.

5. A measuring method to measure biological information, comprising the steps of:
emitting measuring light by a light emitter;
receiving scattering light of the measuring light from a measured part by a light receiver including a plurality of light receiving areas; and
generating biological information by a controller based on output from the plurality of light receiving areas.
